(19) United States
(12) Patent Application Publication
(10) Pub. No.: US 2025/0040870 A1
(43) Pub. Date: Feb. 6, 2025

(54) PHOTOACOUSTIC MONITORING OF ANGIOGENESIS FOR PREDICTING RESPONSE TO THERAPY IN HEALING WOUNDS

(71) Applicant: The Regents of the University of California, Oakland, CA (US)

(72) Inventors: Yash MANTRI, La Jolla, CA (US); Jesse Vincent JOKERST, La Jolla, CA (US)

(21) Appl. No.: 18/715,781

(22) PCT Filed: Dec. 13, 2022
(86) PCT No.: PCT/US22/52662

§ 371 Date: Jun. 3, 2024

Related U.S. Application Data
(60) Provisional application No. 63/290,178, filed on Dec. 16, 2021.

(51) Int. Cl. A61B 5/00

(52) U.S. Cl. CPC A61B 5/445; A61B 5/0095; A61B 5/4848

(57) ABSTRACT

A method for monitoring treatment of a wound includes obtaining a photoacoustic ultrasound image of a wound on a patient. The photoacoustic ultrasound image is processed to extract information that is reflective of a rate of angiogenesis or oxygenation. A degree of wound healing is assessed based at least in part on the extracted information. The wound is treated based at least in part based on the assessed degree of wound healing. The treatment is monitored over time by obtaining and processing additional photoacoustic ultrasound images at subsequent times.

### CROSS REFERENCE TO RELATED APPLICATION

This application claims the benefit for U.S. Provisional Application No. 63/290,178, filed Dec. 16, 2021, the contents of which are incorporated herein by reference.

### GOVERNMENT FUNDING

This invention was made with government support under AG065776 awarded by the National Institutes of Health. The government has certain rights in the invention.

### BACKGROUND

Chronic wounds are a major health problem, but there are no tools to diagnose these wounds before they have erupted and/or evaluate deep tissue response to therapy. Chronic wounds cost the United States medical infrastructure up to $100B/year with a single diabetic ulcer costing nearly $50,000—these numbers will increase as the population ages. To decrease costs and improve quality of life, the community needs tools to predict and monitor response to therapy. Unfortunately, conventional methods are primarily based on visual inspection and cannot see beneath the skin surface—3D mapping of physiology deep into the wound bed could better stratify wound risk and guide therapy but such tools do not exist. While the Braden/Norton scales and transcutaneous oximetry (TCOM) have shown promise, these systems offer an ensemble assessment of the affected area with no spatial details on the wound boundaries, wound depth, and interaction of wound with healthy tissue. Thus, the development of tools to map and measure imaging markers associated with wound risk and treatment response could have a major positive impact for patients with chronic wounds or at risk of developing such wounds.

### SUMMARY

Photoacoustic (PA) ultrasound (US) is a non-invasive, hybrid imaging modality that can solve these major limitations. PA relies on the contrast generated by hemoglobin in blood which allows it to map local angiogenesis, tissue perfusion and oxygen saturation-all critical parameters for wound healing. This work evaluates the use of PA-US to monitor angiogenesis and stratify patients responding vs. not-responding to therapy. We imaged 19 patients with 22 wounds once a week for at least three weeks. Our findings suggest that PA imaging directly visualizes angiogenesis. Patients responding to therapy showed clear signs of angiogenesis and an increased rate of PA increase (p=0.002). These responders had a significant and negative correlation between PA intensity and wound size. Hypertension was correlated to impaired angiogenesis in non-responsive patients. The rate of PA increase and hence the rate of angiogenesis was able to predict healing times within 30 days from the start of monitoring (power=88%, alpha=0.05) This early response detection system could help inform management and treatment strategies while improving outcomes and reducing costs.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### BRIEF DESCRIPTION OF THE DRAWINGS

FIGS. 1A-1R show results of photoacoustic (PA) imaging monitoring of angiogenesis in a healing wound.

FIGS. 2A-2I show results concerning tunneling wounds, wound closure, scar tissue development and angiogenesis.

FIGS. 3A-3I shows results concerning wound progression in a non-responding patient.

FIGS. 4A-4C show results concerning photoacoustic imaging to predict wound healing and response to therapy.

FIG. 5 shows the rate of PA increase per day for the full study period, demonstrating its effectiveness as an imaging biomarker to predict wound healing times, which reduce exponentially as a function of the rate of PA increase (n=22, R²=0.49).

FIGS. 6A-6C are graphs showing wound area vs. time (FIG. 6A), PA intensity vs. time (FIG. 6B) and PA intensity vs. wound area (FIG. 6C) for patient number (PN) 1.

FIGS. 7A-7C are graphs showing wound area vs. time (FIG. 7A), PA intensity vs. time (FIG. 7B) and PA intensity vs. wound area (FIG. 7C) for patient number (PN) 2.

FIGS. 8A-8C are graphs showing wound area vs. time (FIG. 8A), PA intensity vs. time (FIG. 8B) and PA intensity vs. wound area (FIG. 8C) for patient number (PN) 3.

FIGS. 9A-9C are graphs showing wound area vs. time (FIG. 9A), PA intensity vs. time (FIG. 9B) and PA intensity vs. wound area (FIG. 9C) for patient number (PN) 4.

FIGS. 10A-10C are graphs showing wound area vs. time (FIG. 10A), PA intensity vs. time (FIG. 10B) and PA intensity vs. wound area (FIG. 10C) for patient number (PN) 5.

FIGS. 11A-11C are graphs showing wound area vs. time (FIG. 11A), PA intensity vs. time (FIG. 11B) and PA intensity vs. wound area (FIG. 11C) for patient number (PN) 6.

FIGS. 12A-12C are graphs showing wound area vs. time (FIG. 12A), PA intensity vs. time (FIG. 12B) and PA intensity vs. wound area (FIG. 12C) for patient number (PN) 7-WA.

FIGS. 13A-13C are graphs showing wound area vs. time (FIG. 13A), PA intensity vs. time (FIG. 13B) and PA intensity vs. wound area (FIG. 13C) for patient number (PN) 7-WB.

FIGS. 14A-14C are graphs showing wound area vs. time (FIG. 14A), PA intensity vs. time (FIG. 14B) and PA intensity vs. wound area (FIG. 14C) for patient number (PN) 8.

FIGS. 15A-15C are graphs showing wound area vs. time (FIG. 15A), PA intensity vs. time (FIG. 15B) and PA intensity vs. wound area (FIG. 15C) for patient number (PN) 9-WA.

FIGS. 16A-16C are graphs showing wound area vs. time (FIG. 16A), PA intensity vs. time (FIG. 16B) and PA intensity vs. wound area (FIG. 16C) for patient number (PN) 9-WB.

FIGS. 17A-17C are graphs showing wound area vs. time (FIG. 17A), PA intensity vs. time (FIG. 17B) and PA intensity vs. wound area (FIG. 17C) for patient number (PN) 9-WC.

FIGS. 18A-18C are graphs showing wound area vs. time (FIG. 18A), PA intensity vs. time (FIG. 18B) and PA intensity vs. wound area (FIG. 18C) for patient number (PN) 10.

FIGS. 19A-19C are graphs showing wound area vs. time (FIG. 19A), PA intensity vs. time (FIG. 19B) and PA intensity vs. wound area (FIG. 19C) for patient number (PN) 11.

FIGS. 20A-20C are graphs showing wound area vs. time (FIG. 20A), PA intensity vs. time (FIG. 20B) and PA intensity vs. wound area (FIG. 20C) for patient number (PN) 12.

FIGS. 21A-21C are graphs showing wound area vs. time (FIG. 21A), PA intensity vs. time (FIG. 21B) and PA intensity vs. wound area (FIG. 21C) for patient number (PN) 13.

FIGS. 22A-22C are graphs showing wound area vs. time (FIG. 22A), PA intensity vs. time (FIG. 22B) and PA intensity vs. wound area (FIG. 22C) for patient number (PN) 14.

FIGS. 23A-23C are graphs showing wound area vs. time (FIG. 23A), PA intensity vs. time (FIG. 23B) and PA intensity vs. wound area (FIG. 23C) for patient number (PN) 15.

FIGS. 24A-24C are graphs showing wound area vs. time (FIG. 24A), PA intensity vs. time (FIG. 24B) and PA intensity vs. wound area (FIG. 24C) for patient number (PN) 16.

FIGS. 25A-25C are graphs showing wound area vs. time (FIG. 25A), PA intensity vs. time (FIG. 25B) and PA intensity vs. wound area (FIG. 25C) for patient number (PN) 17.

FIGS. 26A-26C are graphs showing wound area vs. time (FIG. 26A), PA intensity vs. time (FIG. 26B) and PA intensity vs. wound area (FIG. 26C) for patient number (PN) 18.

FIGS. 27A-27C are graphs showing wound area vs. time (FIG. 27A), PA intensity vs. time (FIG. 27B) and PA intensity vs. wound area (FIG. 27C) for patient number (PN) 19.

FIGS. 28A-28C are graphs illustrating that the effect of age (years) (FIG. 28A), systolic blood pressure (mm of Hg) (FIG. 27B), and body mass index (BMI) (FIG. 28C) for the entire cohort on the rate of PA change shows no significant correlation.

FIGS. 29A-29C are graphs illustrating the effect of age (years) (FIG. 29A), systolic blood pressure (mm of Hg) (FIG. 29B), and body mass index (BMI) (FIG. 29C) for the responders on the rate of PA change.

FIGS. 30A-30F show 3D mappings of angiogenesis.

### DETAILED DESCRIPTION

### Introduction

Ultrasound (US) imaging is non-invasive and rapid and can make 3D maps of the wound. US is an affordable, high resolution, sensitive, non-ionizing, and real-time tool for imaging but its use is surprisingly rare in wound care despite being ideally suited to characterize soft tissue and bone surfaces. Recently, we reported the use of US to assess wound size in 45 patients. (see Mantri Y. et al., Point-of-Care Ultrasound as a Tool to Assess Wound Size and Tissue Regeneration after Skin Grafting. Ultrasound in Medicine & Biology 2021). We also performed a longitudinal study of wound healing in patients who received allogenic skin grafts over a 110-day period. We showed that ultrasound imaging can predict wound exacerbation and tissue loss before it is seen by the eye. However, ultrasound alone mostly provides anatomic information: There are few details on perfusion or oxygenation, which are critical to wound formation and wound healing. In contrast photoacoustic (PA) ultrasound is a “light in, sound out” technique versus conventional “sound in, sound out” ultrasound. Contrast in photoacoustic ultrasound is generated by differential absorption of light: hemoglobin and deoxyhemoglobin are common absorbers. Thus, photoacoustic imaging can report tissue oxygenation and tissue perfusion. The same scan also collects standard ultrasound images.

Angiogenesis is the formation of new blood vessels from pre-existing vessels. It is well known that angiogenesis is crucial for wound healing. The new blood vessels carry essential cytokines and oxygen for wound repair. Studies have shown that elevated glucose levels in diabetic patients hinders angiogenesis resulting in diabetic ulcer formation, poor wound healing, and limb loss. Treatment protocols such as hyperbaric oxygen therapy, negative pressure wound therapy, and debridement can promote angiogenesis and improve healing outcomes. Hypertension can impair angiogenesis. Hence, an early angiogenesis detection tool could help direct treatment protocols and drastically improve outcomes. Multi-photon microscopy techniques can visualize angiogenesis in vivo but these have micron-scale depth penetration. PA imaging is ideally suited for this application due to centimeter-scale depth penetration and the contrast generated by hemoglobin in blood vessels. Others have recently demonstrated the use of PA imaging to assess peripheral hemodynamic changes in humans, and thus we were motivated to use photoacoustic imaging to visualize angiogenesis. We show here that the rate of PA signal increase directly reports the rate of angiogenesis. We further show that the rate of PA change could be used to predict time to heal. This could help clinicians make early and better-informed decisions on whether a particular treatment regimen should be continued.

### Materials and Methods

### Patients

Patient inclusion criteria were (i) age >18 years and be able to provide consent; (ii) wounds smaller than 15 cm²; (iii) patients must undergo a minimum of three scans spaced at least one week apart from each other. Exclusion criteria included (i) presence of secondary lesions at the wound site (e.g., melanomas); (ii) blood-borne diseases; (iii) orthopedic implants near the wound site. Twenty-one patients (24 wounds) were recruited for this study at the UCSD Hyperbaric Medicine and Wound Care Center, Encinitas, CA, USA. Two patients were excluded from analysis due to poor US coupling reducing image quality. Table 1 describes the patient demographic.

TABLE 1
Patient demographic distribution.

| Category | Distribution | |
|---|---|---|
| Total participants | 19 | |
| Number of scanning events range | 3-11 | scans |
| Monitoring time range for | 21-112 | days |
| individual patients | | |

| | |
|---|---|
| Average age (years) | 63.5 ± 14.4 |
| Sex (Male/Female) | 11/8 |
| Body mass index (kg/m²) | 29.4 ± 7.4 |
| Diabetes (Y/N) | 8/11 |
| Hypertension (Y/N) | 12/7 |
| Smoker (Y/N) | 8/11 |

All patients were scanned during a routine wound care visit. Patients were scanned once a week for at least three weeks. C.A.A. was the independent wound specialist and decided the treatment regimen for all patients blinded to the results of the scan. Before scanning, all wound dressings were removed per standard of care, and the wound area was cleaned using sterile saline. Surrounding healthy tissue was cleaned using alcohol swabs to prevent infection. A sterile CIV-Flex transducer cover (Product no. 921191, AliMed Inc., Dedham, MA, USA) was used for every scan to prevent cross contamination.

### Photoacoustic—Ultrasound Imaging

We used a commercially available LED-based photoacoustic imaging system (AcousticX from Cyberdyne Inc., Tsukuba, Japan). The AcousticX system uses two LED-arrays operating at 850 nm, pulse width 70 ns, and 4 kHz repetition rate. The 128-element linear ultrasound transducer operates at a central frequency of 7 MHz, bandwidth of 80.9%, and a 4 cm field of view. We used a custom hydrophobic gel pad from Cyberdyne Inc. and sterile ultrasound coupling gel (Aquasonic 100, Parker Laboratories Inc., Fairfield NJ, USA) for coupling with the wound surface. All images were acquired at 30 frames/s.

All wounds were scanned in a single sweep from inferior healthy tissue to wound region to superior healthy tissue. All scans were performed by hand, and thus frame alignment between scans was extremely difficult. Due to limitations in image exportation from the software, and to minimize misalignment effects between scans, we chose three representative frames from the central region of the wound for processing. Clinicians also report size and healing assessment from the wound's center. Furthermore, we matched the underlying bone pattern to compare similar spots over time. Y.M. acquired all the images.

### Image Processing

All frames were reconstructed and visualized using the AcousticX software (Cyberdyne Inc.; Version 2.00.10). We exported 8-bit PA, B-mode, and overlayed coronal cross-section images. The images were further processed using Fiji, an ImageJ extension, version 2.1.0/1.53c. Frames with incomplete US coupling were excluded from analysis. Data was plotted using Prism version 9.0.0. We drew custom regions-of-interest (ROIs) for every frame. We quantified changes in wound area, tissue regeneration, scar tissue development, and photoacoustic intensity as a function of time.

Wound area and tissue regeneration were quantified using a method described in the aforementioned reference to Y. Mantri et al. Briefly, we determined a dynamic baseline US intensity of healthy tissue for each patient. Areas with intensity lower or higher than baseline values were classified as wound and scar tissue respectively. Wound and scar area were measured using custom ROIs that fit the above classification criteria. Changes in PA intensity was measured using rectangular ROIs (4 cm wide×1 cm deep). ROIs were drawn under the dermal layer (first 2 mm) hence avoiding PA signal from scabs and hyperpigmented regions of the skin. PA ROIs was made larger to cover the entire field of view of the transducer (4 cm). This is important so we did not miss any signs of angiogenesis from the periphery of the wound. ROIs for PA intensity measurements were also kept constant for all patients eliminating any concerns of inter-rater reliability. All US and PA quantification were carried out on the same frames.

### Statistics

We measured wound area and PA intensity in three frames for each scan. The error bars in each figure represent the standard deviation within these three frames. A simple linear regression was fit to the data measuring changes in imaging markers over time; 95% confidence intervals for these fits are shown in each figure. Furthermore, we plotted the rate of PA change per day vs. the healing time for the study population and fit a one-phase exponential decay curve to it. We used a Pearson correlation test to determine the correlation between the time to heal (days) versus rate of PA increase comparing the null hypothesis that there is no correlation versus there is a negative correlation between these two variables. The statistical analyses were conducted at alpha=0.05. A power analysis was also performed on this data. We used a two-tailed Fisher's exact test to look for significant differences in clinical features between responders and non-responders. An area under the curve-receiver operating characteristic (AUC-ROC) analysis was performed to study the classification of therapeutic responders vs non-responders.

### FIGURES

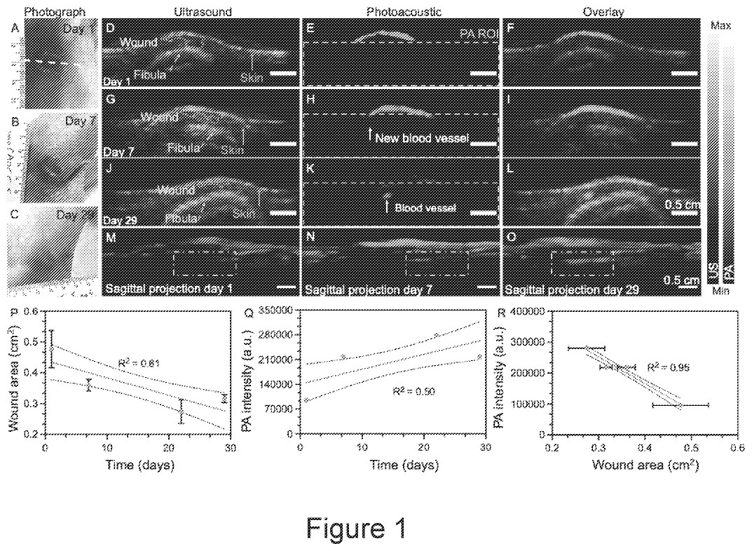

FIGS. 1A-1R show results of photoacoustic (PA) imaging monitoring of angiogenesis in a healing wound. PN1 is an 82-year-old female presenting with a chronic, left posterolateral ankle ulcer. FIGS. 1A-1C are photographs showing the wound on days 1, 7, and 29 of the study. White dotted line indicates the imaging plane. FIGS. 1D-1F, 1G-1I, and 1J-1 show US, PA, and overlayed images of the wound on days 1, 7, and 29, respectively. The dotted (wound) region of interest (ROI) on the US outlines the wound. Solid and dashed arrows mark the skin surface and fibula respectively. White solid arrows (FIGS. 1H and 1K) show new blood vessel formation i.e., angiogenesis. The dashed outline marks the ROI for PA intensity measurement. FIGS. 1M-10 show the sagittal maximum intensity projection of the wound on days 1, 7 and 29 showing new blood vessels invading the wound bed (Dashed-dot box). All scale bars are 0.5 cm. FIG. 1P shows a negative correlation between wound area and time suggests wound closure (R²=0.61). FIG. 1Q shows a significant positive correlation between PA intensity and time, suggesting angiogenesis within the wound bed (R²=0.50). The rate of PA increase is 4217±1336 intensity a.u./day. FIG. 1R shows that the PA intensity increases linearly as the wound heals, suggesting that angiogenesis is correlated to wound closure (R²=0.95). Scale bars represent 0.5 cm. Error bars represent standard deviation in 3 representative frames from the center of the wound. Error bars for PA intensity in FIGS. 1Q and 1R are too small to be shown.

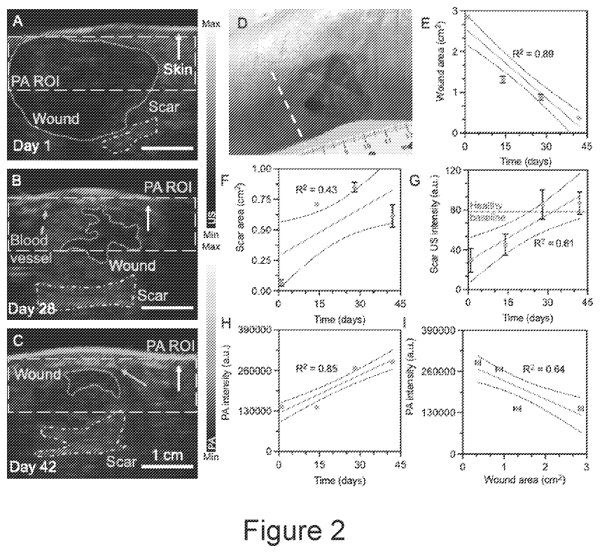

FIGS. 2A-2I show results concerning tunneling wounds, wound closure, scar tissue development and angiogenesis. FIGS. 2A-2C show US-PA overlays of the wound on days 1, 28, and 42 of the study. Dotted, dashed-dot, and dashed lines in FIGS. 2A-2C represent wound, scar area, and PA ROI respectively. Solid white and dashed grey arrows represent skin surface and blood vessels respectively. FIG. 2D is a photographic image of wound in the left posterior ankle region. There is significant tunneling of the wound (not seen by eye). The dashed line in FIG. 2D indicates the relative imaging plane for FIGS. 2A-2C. FIG. 2E shows that an 87% wound contraction is seen within 42-days. In FIGS. 2F-2G scar tissue development is seen as hyperechoic regions at the wound bed. FIG. 2H shows a significant increase in PA intensity over time, indicating angiogenesis. FIG. 2I shows a negative correlation between PA intensity and wound area, suggesting angiogenesis results in wound closure. Scale bars represent 1 cm. Error bars represent standard deviation in three frames at the center of the wound. Error bars for PA intensity in FIGS. 2H and 2I are too small to be shown.

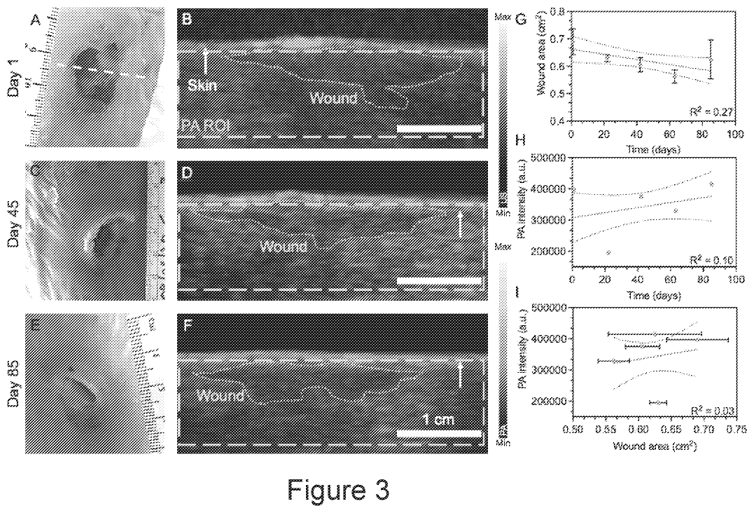

FIGS. 3A-3I shows results concerning wound progression in a non-responding patient. FIGS. 3A-3B, 3C-3D, and 3E-3Fs are photographs, and US—PA overlays on days 1, 45, and 85 of the study respectively. No significant changes in wound size can be seen in the pictures and US scans. Dotted and dashed lines outline the wound region and PA ROI used for processing. White solid arrow marks the skin surface. White dashed line in FIG. 3A marks the imaging plane. FIG. 3G shows that the mean wound area is reduced by 9.4% in the 85-day period, but this change was not statistically significant (R²=0.27). FIG. 3H shows that the PA intensity in the wound increased at a rate of 807.7±706.7 intensity a.u./day, R²=0.10, showing no significant correlation versus time. This suggests the absence of angiogenesis and the need for a different therapeutic approach. In FIG. 3I, the plot of PA intensity vs. wound area shows no significant correlation (R²=0.03). Scale bars represent 1 cm. Error bars represent standard deviation in three frames at the center of the wound. Error bars for PA intensity in FIGS. 3H and 3I are too small to be shown.

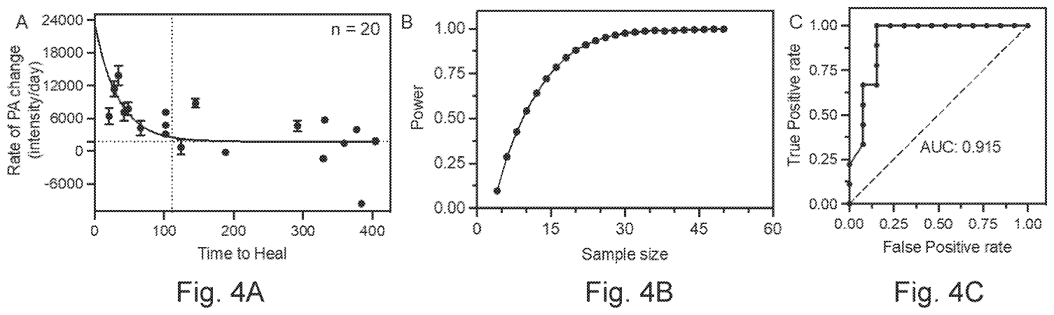

FIGS. 4A-4C show results concerning photoacoustic imaging to predict wound healing and response to therapy. FIG. 4A shows that the rate of PA increase per day within the first 30 days is an effective imaging marker to predict wound healing time. Healing times reduce exponentially as a function of the rate of PA increase (n=20). This could help classify patients as responders (grey shaded area) vs. non-responders to a particular therapy. PA could help in the early identification of non-responders allowing clinicians to change their therapeutic approach and improve outcomes. FIG. 4B is a power analysis using the data used in FIG. 4A, showing that 80% power with an alpha of 0.05 was achieved at n=17. At n=20 the power was 88%, p=0.0009. Error bars in panel A represent standard error in the rate of PA change for each patient. FIG. 4C shows true positive rate vs. the false positive rate.

### Results

Nineteen patients with 22 wounds were analyzed in this study. All patients underwent at least three scans spaced one week apart. We measured changes in wound area, PA intensity, and scar tissue formation over time. Table 2 lists all the wound and relevant patient information. Nine wounds showed response to therapy. Table 3 shows the clinical features of therapeutic responders and non-responders. Responders were patients who healed within 111 days. Hypertension was significantly (p=0.0001) responsible for delayed healing. We noted no significant difference in other clinical features (age, sex, diabetes, smoking, body mass index (BMI), heart rate, blood pressure, and oxygen saturation) between the two groups. Extreme cases of wounds that had a swift, delayed and no response to therapy have been highlighted below.

TABLE 2
Patient and wound information.

| | | | Number of | First imaging | | | | |
|---|---|---|---|---|---|---|---|---|
| | | | source | session after | | | | Response |
| | | | (Study | initial | | Healing | Hyper- | to |
| | Age | Sex | period | presentation | | time | tensive | therapy |
| Pat. No. | (yrs) | (M/F) | of days) | (days) | Wound description | (days) | (Y/N) | (Y/N) |
| PN1 | 82 | F | 4 (29) | 18 | Left posterolateral ankle | 66 | N | Y |
| PN2 | 59 | M | 4 (42) | 7 | Left posterior ankle | 292 | Y | N |
| PN3 | 70 | F | 5 (85) | 175 | Left heel | 384 | Y | N |
| PN4 | 70 | M | 6 (112) | 110 | Right Achilles ulcer | 411* | Y | N |
| PN5 | 28 | M | 3 (118) | 490 | Inferior to the left knee | 357 | Y | N |
| PN6 | 77 | M | 11 (110) | 103 | Inferior to the left knee | 124 | Y | N |
| PN7-WA | 71 | M | 3 (21) | 2252 | Left dorsal ankle | 404* | Y | N |
| PN7-WB | 71 | M | 3 (21) | 2252 | Left dorsal heel | 404* | Y | N |
| PN8 | 56 | M | 4 (51) | 32 | Greater toe of right foot | 331 | Y | N |
| PN9-WA | 56 | F | 4 (35) | 47 | Lower anterior left leg | 102 | N | Y |
| PN9-WB | 56 | F | 4 (35) | 47 | Lower medial left leg | 102 | N | Y |
| PN9-WC | 56 | F | 4 (35) | 47 | Lower left leg | 102 | N | Y |
| PN10 | 72 | M | 7 (99) | 229 | Left posterior leg | 329 | Y | N |
| PN11 | 78 | F | 6 (98) | 421 | Lateral left ankle | 188 | Y | N |
| PN12 | 33 | M | 10 (106) | 33 | Lower anterior left leg | 145 | Y | N |
| PN13 | 67 | F | 4 (34) | 32 | Toe of left foot | 34 | N | Y |
| PN14 | 62 | M | 4 (77) | 740 | Left plantar great toe | 377* | Y | N |
| PN15 | 54 | M | 4 (76) | 329 | Mid-lateral right foot | 359 | N | N |
| PN16 | 62 | F | 3 (21) | 35 | Right lower leg | 21 | N | Y |
| PN17 | 82 | F | 4 (28) | 12 | Right lower leg | 28 | N | Y |
| PN18 | 60 | M | 6 (42) | 14 | Right medial ankle | 42 | Y | Y |
| PN19 | 68 | M | 3 (22) | 43 | Right plantar region | 48 | N | Y |

Shaded entries indicate patients who are non-hypertensive and show good response to therapy.
*Indicates patients who were still receiving wound care at the time of submission.
WA, B, and C, denote different wounds on the same patient.

TABLE 3
Clinical features of responders (healing time <111 days)
and non-responders. This data is from 19 patients with 22
wounds. Values are mean ± SD or number of subjects (%).

| | | Non- | |
|---|---|---|---|
| | Responders | Responders | |
| Category | (n = 9 wounds) | (n = 13 wounds) | p values |
| Age (years) | 68.1 ± 10.3 | 60.9 ± 15.1 | 0.31 |
| Sex (male) | 2 (29%) | 9 (75%) | 0.051 |
| Diabetes | 2 (22%) | 5 (38%) | 0.59 |
| Hypertension | 1 (11%) | 11 (85%) | 0.0001* |
| Smoker | 2 (29%) | 4 (31%) | 0.39 |
| BMI (kg/m²) | 28.2 ± 9.6 | 30.5 ± 5.9 | 0.63 |
| Heart rate (bpm) | 74 ± 12 | 92 ± 22 | 0.19 |
| Blood Pressure | 147/80 ± 22/6 | 154/91 ± 30/22 | 0.78 |
| (systolic/ | | | |
| diastolic) | | | |
| (mm of Hg) | | | |
| Oxygen | 98.4 ± 0.97 | 97.7 ± 1.4 | 0.31 |
| saturation (%) | | | |
| Rate of PA | 6698 ± 4217 | 2501 ± 2129 | 0.002* |
| change | | | |
| (Intensity | | | |
| (a.u.)/day) | | | |
| Wound size | 0.931 ± 0.52 | 0.978 ± 0.70 | 0.33 |
| on Day 1of | | | |
| imaging(cm²) | | | |
| Wound size | 0.454 ± 0.35 | 0.509 ± 0.25 | 0.43 |
| on last day | | | |
| of imaging (cm²) | | | |

| | |
|---|---|
| Time of Initiation | Not controlled see Table S2. |
| after first | |
| presentation | |
| (days) | |

*Marks significant difference (p < 0.05).

FIG. 1 shows wound healing and angiogenesis in an 82-year-old female (Subject ID: PN1) presenting with a chronic, left posterolateral ankle ulcer. Patent number 1 (PN1) healed in 66 days. Wound healing was visible via photographs within the first 29 days of treatment (FIGS. 1 A-C). US imaging showed a 33.3% reduction in wound size over 29 days from 0.48 cm²-0.32 cm² (FIG. 1P). The wound area reduced linearly as a function of time (R²=0.61). PA imaging showed the formation of new blood vessels on day 7 (FIG. 1H). PA intensity increased linearly at a rate of 4217±1336 intensity a.u./day as the wound healed (R²=0.50) (FIG. 1Q). A sagittal maximum intensity projection (MIP) of the wound area showed angiogenesis into the wound bed (FIGS. 1 M-O). FIG. 1R shows a negative correlation between wound area and PA intensity (R²=0.95).

FIG. 2 shows the progress of the wound healing indicators in PN2. PN2 was a 59-year-old male presenting with a chronic left ankle ulcer following a severed Achilles tendon repair surgery. PN2 underwent three scans (day 1, 14, 28, 42) and took 292 days to heal. Photographs show tunneling of the wound under healthy surface tissue superior to the wound (FIG. 2D). Blue dotted lines represent the imaging plane. It is important to note that tunneling wounds cannot be assessed non-invasively by the eye. FIG. 2A-C, show wound progression over the 42-day study period. The wound tunnel showed 87% contraction by day 42 and wound area showed a strong negative correlation with treatment time (R²=0.89) (FIG. 2E). More importantly, this patient showed the development of scar tissue by day 28 that was also mentioned in the doctor's notes. Tissue was considered scarred if the mean US intensity was higher than healthy tissue baseline. Scar area was measured using custom ROIs with maximum size fitting the above criteria. Scar area and intensity increased linearly as a function of time (R²=0.43 and 0.61 respectively) (FIGS. 2 F-G). PA intensity in the wound area increased linearly at a rate of 4078±534 intensity a.u./day, R²=0.85 (FIG. 2H). PA intensity was negatively proportional to wound area (R²=0.64) (FIG. 2I).

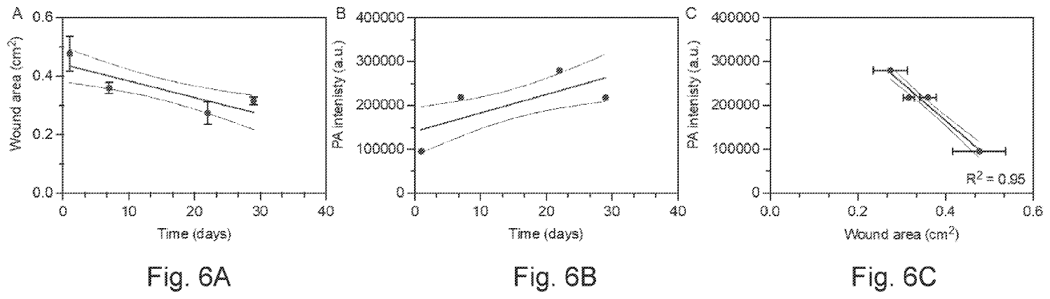

FIG. 3 shows progression in a non-healing wound. Subject PN3 was a 70-year-old female presenting with a stage III pressure ulcer on her left heel. PN3 took over 384 days to heal and was still receiving wound care during the preparation of this manuscript. PN3 underwent five scans over an 85-day period, and received standard wound care decided by the attending physician C.A.A. Photographs showed no visible contraction of the ulcer (FIGS. 3A, C, and E). US imaging showed a 9.4% reduction in wound size over 85-days (FIG. 3G). PA intensity increased by 4.2% during the same interval (FIG. 3H). No clear signs of angiogenesis were visible at any point of the study. There was no significant correlation between PA intensity and wound area, R²=0.03 (FIG. 3I). The data for individual patients showing changes in wound area, PA intensity as a function of time, and PA intensity vs. wound area are shown in the supporting information (FIGS. 6-27).

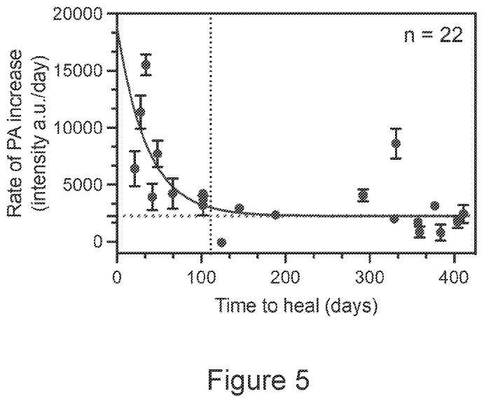

FIG. 4 shows population wide analysis for 17 patients with 20 wounds within the first 30 days of monitoring. The rate of PA increase was derived from the plot of PA intensity vs. time for each wound. Error bars represent the standard error of the slope. Healing times were noted from the patient charts as reported by the clinic and C.A.A. Two patients had scans more than 30 days apart and hence were dropped from the analysis in FIG. 4A. The full-length monitoring period for all patients can be found in the FIG. 5 that shows a similar trend as in FIG. 4A. The minimum amount of time needed to classify a patient is 30 days. A one-phase exponential decay curve was fit to the data with an R²=0.76. The plateau was calculated to be 1738 intensity a.u./day. Wounds were classified into responders and non-responders using rate of PA change and healing time; 111 days was used as a cutoff for this classification based on previously reported values in literature. The green shaded region (n=9 wounds) in FIG. 4A shows wounds classified as responders to therapy. The other 11 wounds were classified as non-responders. A power analysis using the data in FIG. 4A showed 80% power with an alpha of 0.05 with 17 wounds. The power was 80% with p=0.0009 for 20 wounds (FIG. 4B). Hence, the sample size was statistically sufficient to draw clinically significant conclusions. FIG. 4C shows the AUC-ROC curves for discriminating responders vs. non-responders. Responders were patients with a rate of PA intensity increase greater than 1738 intensity a.u./day and healing time less than 111-days. The AUC-ROC value is 0.915, which is higher than other reported wound-prediction techniques (Table 4).

TABLE 4
PA imaging has the highest AUC values compared to other
commonly used wound healing prediction techniques.

| Prediction technique | AUC | Reference |
|---|---|---|
| Photoacoustic imaging | 0.915 | Current work |
| Transcutaneous oxygen monitoring | 0.805 | (59) |
| Ankle brachial index | 0.630 | (60) |
| Toe brachial index | 0.560 | (60) |
| Multispectral imaging | 0.700 | (61) |
| Toe blood pressure | 0.760 | (62) |
| Demographics only | 0.556 | (48) |
| Demographic + clinical characteristics | 0.605 | (48) |
| Demographic + clinical characteristics + | 0.712 | (48) |
| wound characteristics | | |

### Discussion

### Imaging Parameters

PA imaging is ideally suited to monitor local angiogenesis, perfusion, and oxygen saturation: These are all key parameters for wound healing. Multiple studies have shown the use of PA tomography and microscopy to visualize the skin surface, superficial blood vessels, and blood flow with exceptional spatial resolution (<100 μm, lateral resolution). The LED-based PA system used in this study has much lower spatial resolution and fluence but is also less expensive and more robust/portable compared to conventional high energy laser-based systems. It employs low-energy LED illumination operating under the maximum permissible exposure limit (2-9 μJ/cm²) with a lateral resolution between 550-590 μm. Hand-held scans using the LED-based PA system allows easy mapping of wounds on contoured surfaces such as the ankle, thus making it ideal to visualize angiogenesis in complex wounds. The 850-nm excitation used in this study falls within the biological optical window and maximizes depth penetration while maintaining a relatively high signal-to-noise ratio (˜35 dB). Limitations of this LED-based system include a small cache: The system acquires 500-1500 frames per scan but the processing software only exports 180 representative frames per scan (1 exported frame for every 8 acquired frames). Hence, there is a large loss of data unless one scans multiple small areas separately. The image exportation limited us to analyze only three representative frames from the center of the wound. Of course, more generally, any suitable PA imaging system may be employed to perform the methods and techniques described herein, which are not limited the aforementioned system used in this study.

One major strength of the study was that all image processing was carried out by a single individual who was blinded to the study. We used carefully considered criteria to define wound vs. scar vs. healthy tissue. Areas were classified as wound or scar tissue if the mean US intensity was lower or higher than healthy tissue baseline, respectively. Custom drawn ROIs analysis can be extremely subjective but we have shown good inter-rater reliability (mean bias 4.4%) in our previous work that used US to quantify tissue regeneration and wound closure in skin grafted patients. The PA intensity was quantified using a rectangular ROI measure 4 cm wide and 1 cm deep and excluding the skin surface. We used the integrated density measurement which adds the intensity of all the pixels in the ROI instead of mean PA intensity. The use of integrated density reduces the effects of poor coupling, if any and provides an absolute value of PA intensity. The PA intensity ROI was maintained constant for all patients, eliminating concerns of subjectivity, and interferences due to skin tone.

### Clinical Significance

It is well established that angiogenesis is critical for wound healing. New blood vessels formed during the healing process deliver key cytokines and oxygen that reshape the wound matrix and result in wound closure. Hence, angiogenesis can be a key imaging marker to predict response to therapy. The Centers for Medicare and Medicaid Services (CMS) in the United States re-evaluates coverage after 30 days from initial patient encounter. Patients needing advanced therapies need to be certified by the attending physician to enter a comprehensive plan of care in the medical record. A recent high-powered study in 620,356 wounds showed that demographics, wound and clinical assessment could be used to predict wound healing in 84 days (AUC=0.712, Table 2). But this is above the 30-day re-evaluation time limit set by CMS.

An important main clinical significance of this study is the ability to classify patients according to their response within 30 days from the start of therapy which aligns with the coverage re-evaluation time from CMS. Compared to other commonly employed techniques such as ankle brachial index, TCOM, etc., PA imaging is the best predictor for wound healing (AUC=0.915, Table 2). PA classification could allow wound specialists to change their course of treatment if the wound is not responding to conventional treatment protocols. This would in turn improve outcomes, reduce amputations, healing time, and costs.

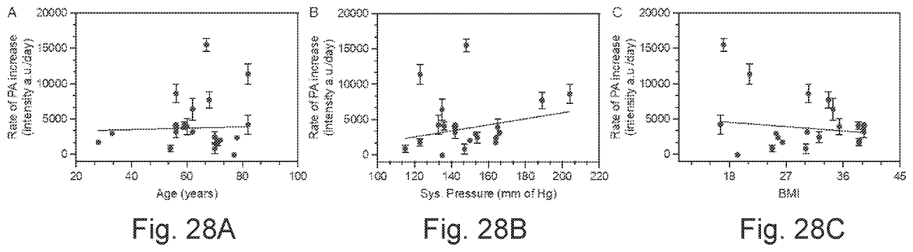

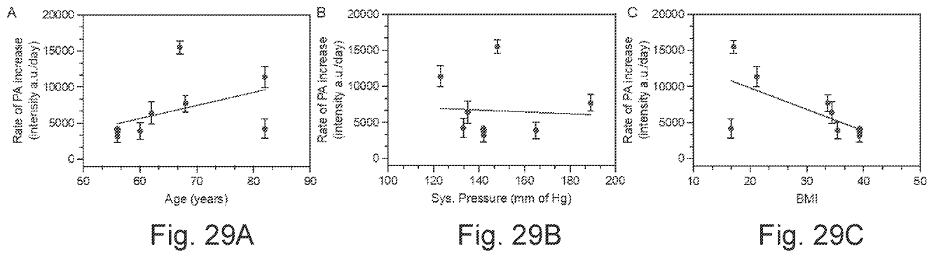

The rate of PA change is indicative of the rate of angiogenesis in the wound bed. The MIPs (FIGS. 1M-1O and FIG. 29) confirms the formation of new blood vessels into the wound bed. Responding patients had a mean rate of PA change 6698±4217 intensity (a.u.)/day that was significantly higher (p=0.002) than non-responders (2501±2129 intensity (a.u.)/day). Within the responding group, higher age and lower BMI were related to an increased rate of PA change (FIG. 28). Age negatively impacts angiogenesis hence the age correlation is unexpected. The difference in treatment regimens like the use of cellularized tissue products to accelerate tissue regeneration could explain the age correlation. Blood pressure had no significant effect on the rate of PA change. Hypertension, diabetes, and smoking are also known to impair angiogenesis and hence wound repair. The effect of hypertension on wound healing is visible in this cohort (Table 1): 12 of the 13 non-responsive wounds were hypertensive (92%), but only 1 of the 9 responsive wounds were hypertensive (14%). Hence non-hypertensive patients are more likely to develop new blood vessels and positively respond to therapy. Clinical factors alone can be used as a classifier but the use of PA imaging significantly improves prediction (Table 2). A larger patient cohort could better illustrate the role of other risk factors that impair healing.

Traditionally, clinicians use surface cues such as color and presence of devitalized tissue to assess wound health. In some cases, wound tunneling or cavitation can lengthen healing times and cause significant discomfort. Conventionally, probing tools are used to measure tunneling depth. Probing is invasive and can lead to further tissue injury. Accurately and safely assessing tunneling wounds is therefore quite difficult visually. PN2 presents as an ideal example of a tunnelling wound to show the power of imaging over conventional wound assessment methods. The US was not only able to measure wound reduction (87% in 42 days), but also monitor scar tissue formation in the wound bed. Scar tissue presents as hyperechoic regions on the US due to its high fibrotic nature. The addition of PA imaging allows us to visualize angiogenesis around the healing wound. Angiogenesis can be clearly seen in FIG. 2B-2C sandwiched in between the wound and skin surface. Deeper blood vessels can be seen on the US in FIG. 2C, but these have very low PA signal due to reduced light penetration through tissue. The presence of a sterile sleeve between the transducer and skin surface also enhances light scattering, further reducing penetration depth. Using a higher wavelength of light could help visualize deeper vessels. The longer healing time compared to PN1 with similar rate of PA change can be attributed to the larger wound size, tunneling, and a different treatment regimen compared to PN1. PNIs wound was limited to skin breakdown whereas PN2s wound had full thickness soft tissue involvement.

Secondary trauma, insufficient off-loading, poor wound dressing practices, and poor patient compliance can significantly impair wound healing and increase healing time. Nevertheless, with 88% power in our study, we believe there is enough statistical significance to draw clinically relevant conclusions from this PA data. Future work in this field will look at employing oximetry-based PA measurements to measure local oxygen tension within the wound. It would also be interesting to study how PA imaging performs in conjunction with other prediction tools. The specialty of hyperbaric medicine could potentially benefit from this study. Such knowledge about oxygenation could potentially improve the use of hyperbaric oxygen treatment, indicating whether it should be initiated, continued, or halted. Patients not responding to therapy can then be more efficiently directed to other wound treatment interventions or therapeutic modalities. Furthermore motion-compensation and deep learning algorithms could improve image stability, quality, and streamline image processing.

In summary, angiogenesis is a key imaging marker for wound healing. PA-US imaging can be used to measure wound size, rate of angiogenesis, and scar tissue formation. A study of 19 patients with 22 wounds revealed that there is an inverse correlation between wound area and PA intensity. An increase in PA intensity correlates with wound closure due to the formation of new blood vessels. 3D MIP images confirmed blood vessel infiltration into the wound bed. Non-healing wounds showed no correlation between PA intensity and wound area. A higher rate of PA increase was associated with an exponential reduction in healing times. Finally, PA imaging could be used to classify therapy responders and non-responders within 30-days from the start of treatment. With an AUC value of 0.915, PA imaging is the best wound prediction technique. This work could have clinical significance in helping doctors make more informed and early decisions about whether treatment should be initiated, continued, altered, or halted.

### ADDITIONAL FIGURES

FIG. 5 shows the rate of PA increase per day for the full study period, demonstrating its effectiveness as an imaging biomarker to predict wound healing times, which reduce exponentially as a function of the rate of PA increase (n=22, R²=0.49).

FIGS. 6A-6C are graphs showing wound area vs. time (FIG. 6A), PA intensity vs. time (FIG. 6B) and PA intensity vs. wound area (FIG. 6C) for patient number (PN) 1.

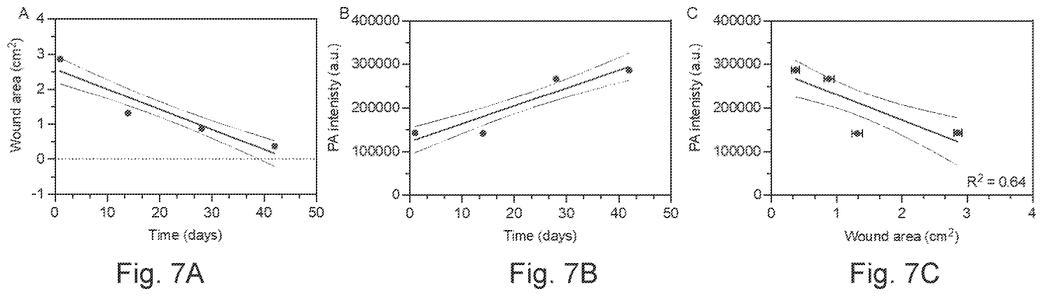

FIGS. 7A-7C are graphs showing wound area vs. time (FIG. 7A), PA intensity vs. time (FIG. 7B) and PA intensity vs. wound area (FIG. 7C) for patient number (PN) 2.

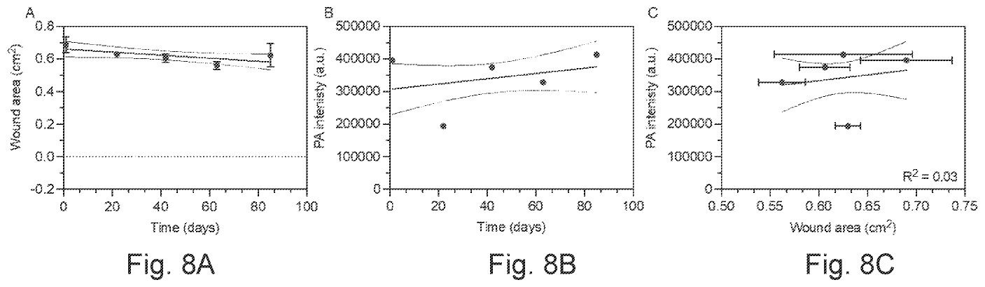

FIGS. 8A-8C are graphs showing wound area vs. time (FIG. 8A), PA intensity vs. time (FIG. 8B) and PA intensity vs. wound area (FIG. 8C) for patient number (PN) 3.

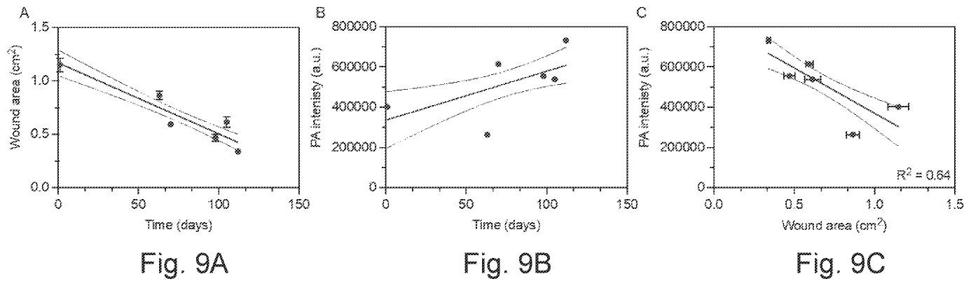

FIGS. 9A-9C are graphs showing wound area vs. time (FIG. 9A), PA intensity vs. time (FIG. 9B) and PA intensity vs. wound area (FIG. 9C) for patient number (PN) 4.

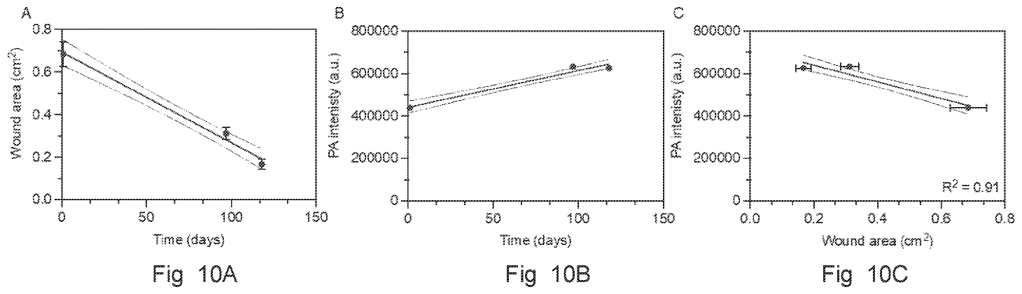

FIGS. 10A-10C are graphs showing wound area vs. time (FIG. 10A), PA intensity vs. time (FIG. 10B) and PA intensity vs. wound area (FIG. 10C) for patient number (PN) 5.

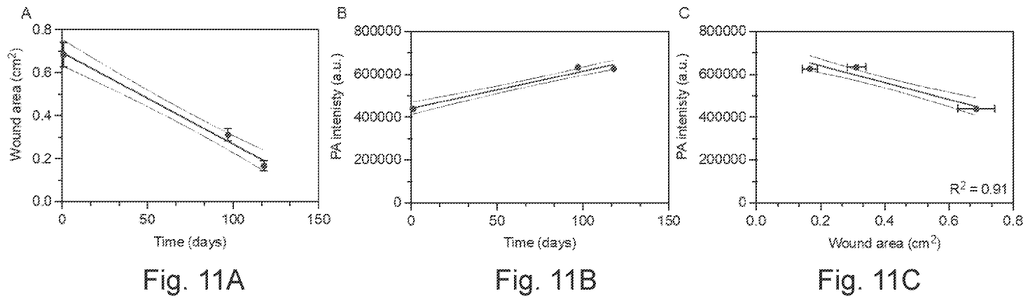

FIGS. 11A-11C are graphs showing wound area vs. time (FIG. 11A), PA intensity vs. time (FIG. 11B) and PA intensity vs. wound area (FIG. 11C) for patient number (PN) 6.

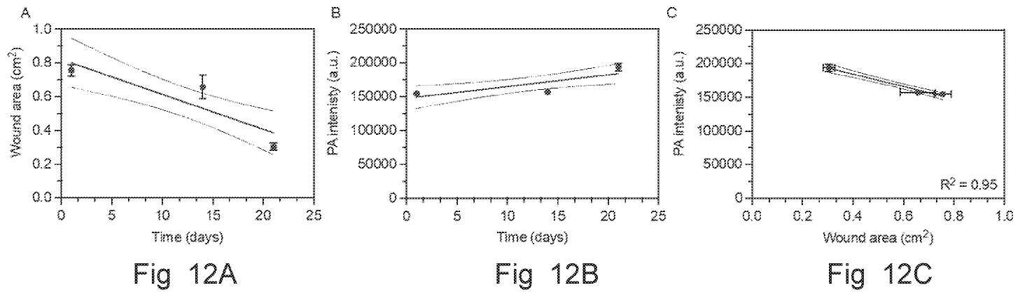

FIGS. 12A-12C are graphs showing wound area vs. time (FIG. 12A), PA intensity vs. time (FIG. 12B) and PA intensity vs. wound area (FIG. 12C) for patient number (PN) 7-WA.

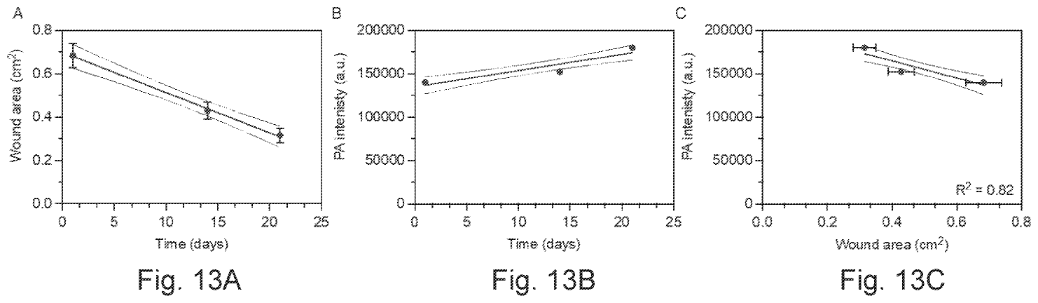

FIGS. 13A-13C are graphs showing wound area vs. time (FIG. 13A), PA intensity vs. time (FIG. 13B) and PA intensity vs. wound area (FIG. 13C) for patient number (PN) 7-WB.

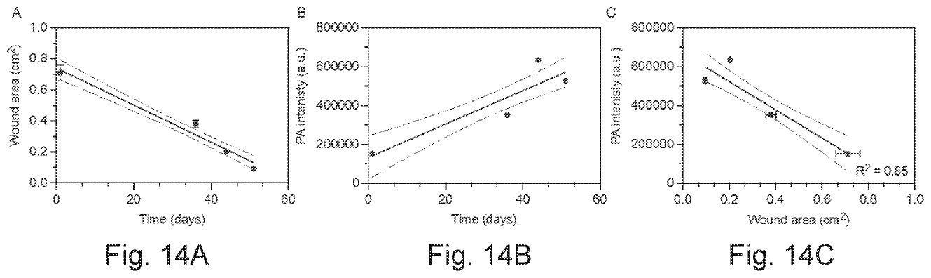

FIGS. 14A-14C are graphs showing wound area vs. time (FIG. 14A), PA intensity vs. time (FIG. 14B) and PA intensity vs. wound area (FIG. 14C) for patient number (PN) 8.

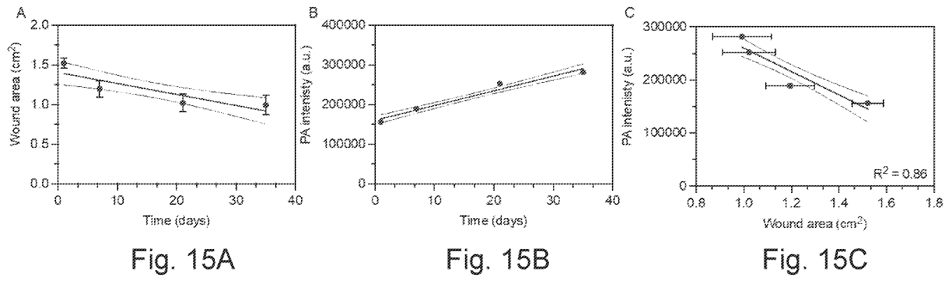

FIGS. 15A-15C are graphs showing wound area vs. time (FIG. 15A), PA intensity vs. time (FIG. 15B) and PA intensity vs. wound area (FIG. 15C) for patient number (PN) 9-WA.

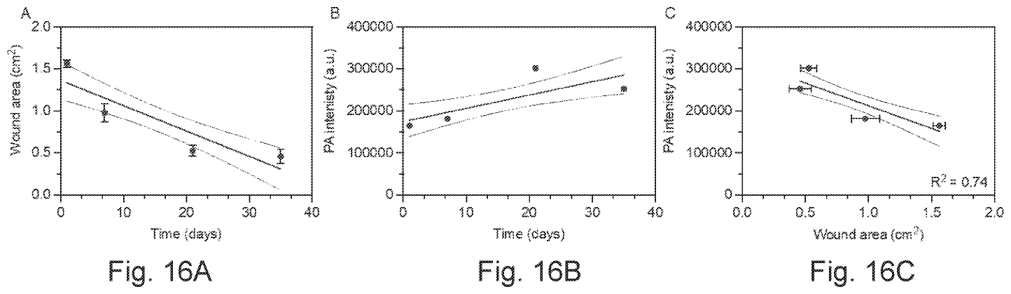

FIGS. 16A-16C are graphs showing wound area vs. time (FIG. 16A), PA intensity vs. time (FIG. 16B) and PA intensity vs. wound area (FIG. 16C) for patient number (PN) 9-WB.

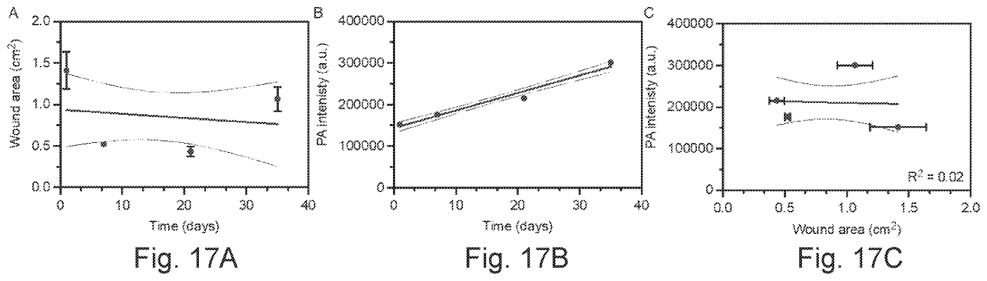

FIGS. 17A-17C are graphs showing wound area vs. time (FIG. 17A), PA intensity vs. time (FIG. 17B) and PA intensity vs. wound area (FIG. 17C) for patient number (PN) 9-WC.

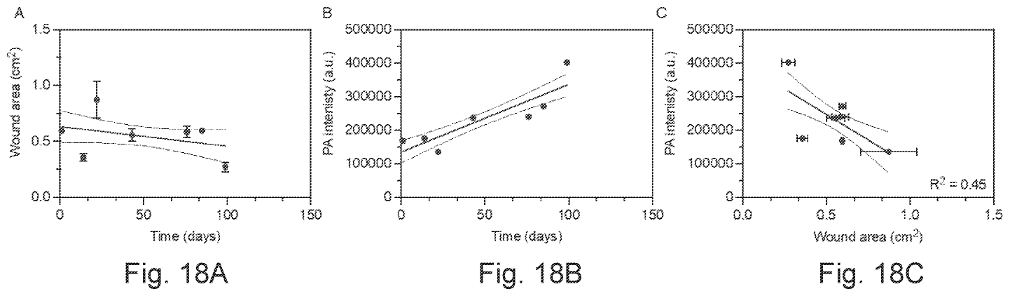

FIGS. 18A-18C are graphs showing wound area vs. time (FIG. 18A), PA intensity vs. time (FIG. 18B) and PA intensity vs. wound area (FIG. 18C) for patient number (PN) 10.

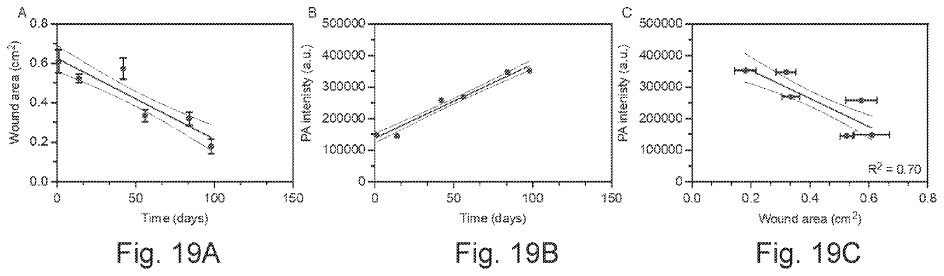

FIGS. 19A-19C are graphs showing wound area vs. time (FIG. 19A), PA intensity vs. time (FIG. 19B) and PA intensity vs. wound area (FIG. 19C) for patient number (PN) 11.

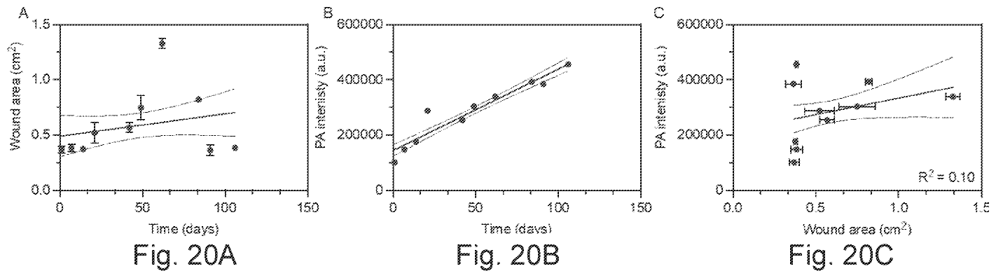

FIGS. 20A-20C are graphs showing wound area vs. time (FIG. 20A), PA intensity vs. time (FIG. 20B) and PA intensity vs. wound area (FIG. 20C) for patient number (PN) 12.

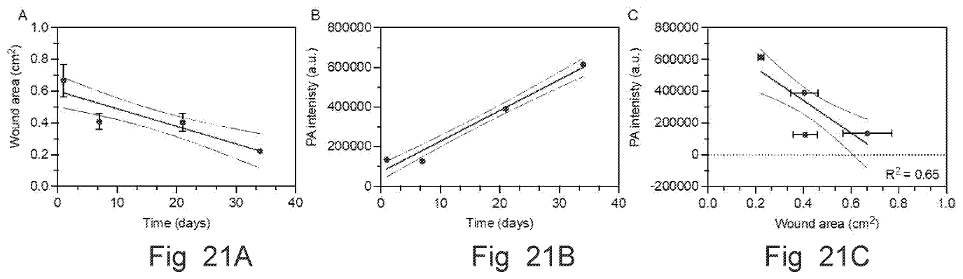

FIGS. 21A-21C are graphs showing wound area vs. time (FIG. 21A), PA intensity vs. time (FIG. 21B) and PA intensity vs. wound area (FIG. 21C) for patient number (PN) 13.

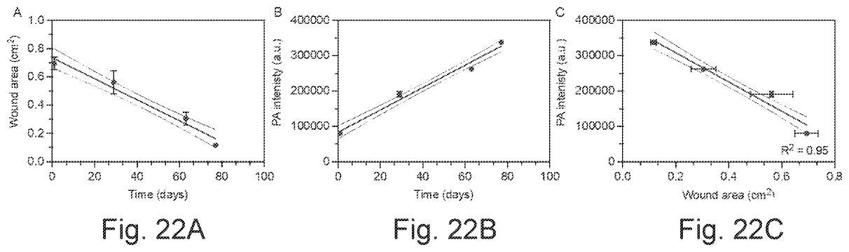

FIGS. 22A-22C are graphs showing wound area vs. time (FIG. 22A), PA intensity vs. time (FIG. 22B) and PA intensity vs. wound area (FIG. 22C) for patient number (PN) 14.

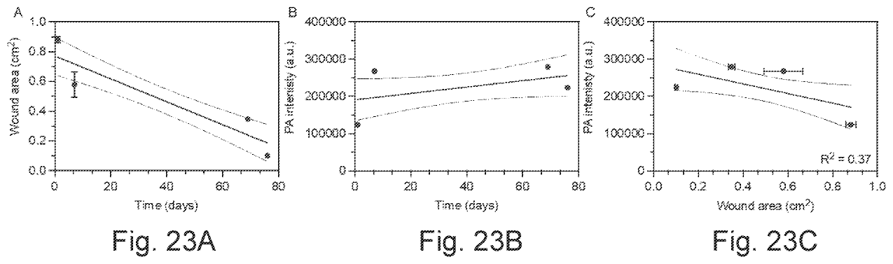

FIGS. 23A-23C are graphs showing wound area vs. time (FIG. 23A), PA intensity vs. time (FIG. 23B) and PA intensity vs. wound area (FIG. 23C) for patient number (PN) 15.

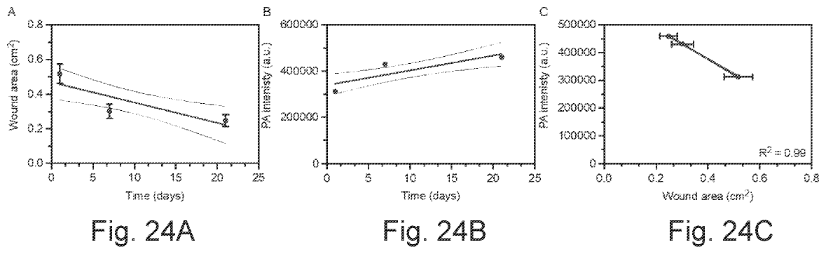

FIGS. 24A-24C are graphs showing wound area vs. time (FIG. 24A), PA intensity vs. time (FIG. 24B) and PA intensity vs. wound area (FIG. 24C) for patient number (PN) 16.

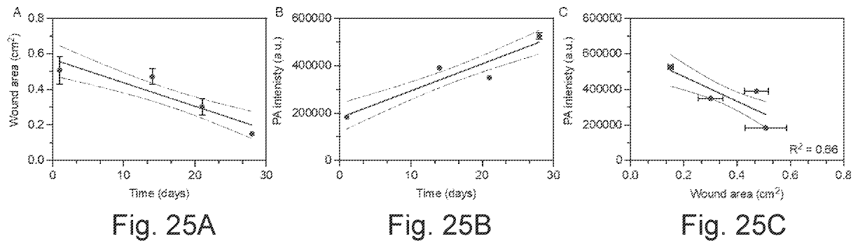

FIGS. 25A-25C are graphs showing wound area vs. time (FIG. 25A), PA intensity vs. time (FIG. 25B) and PA intensity vs. wound area (FIG. 25C) for patient number (PN) 17.

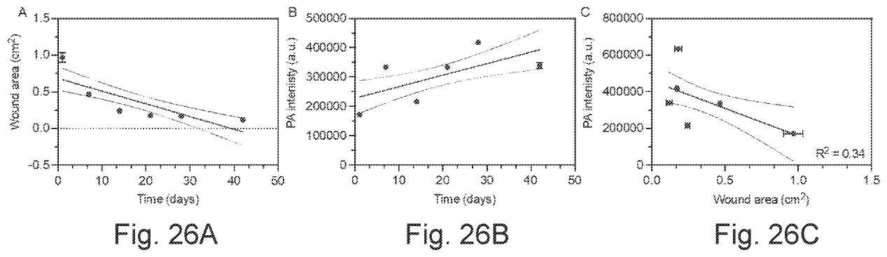

FIGS. 26A-26C are graphs showing wound area vs. time (FIG. 26A), PA intensity vs. time (FIG. 26B) and PA intensity vs. wound area (FIG. 26C) for patient number (PN) 18.

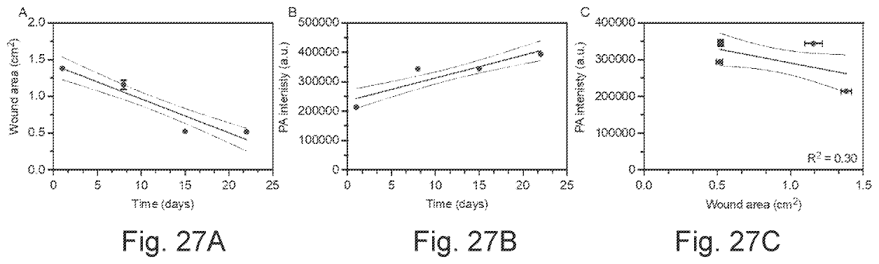

FIGS. 27A-27C are graphs showing wound area vs. time (FIG. 27A), PA intensity vs. time (FIG. 27B) and PA intensity vs. wound area (FIG. 27C) for patient number (PN) 19.

FIGS. 28A-28C are graphs illustrating that the effect of age (years) (FIG. 28A), systolic blood pressure (mm of Hg) (FIG. 27B), and body mass index (BMI) (FIG. 28C) for the entire cohort on the rate of PA change shows no significant correlation.

FIGS. 29A-29C are graphs illustrating the effect of age (years) (FIG. 29A), systolic blood pressure (mm of Hg) (FIG. 29B), and body mass index (BMI) (FIG. 29C) for the responders on the rate of PA change. Higher age and lower BMI are related to increased rate of change in PA intensity. Blood pressure had no significant effect on the rate of PA change.

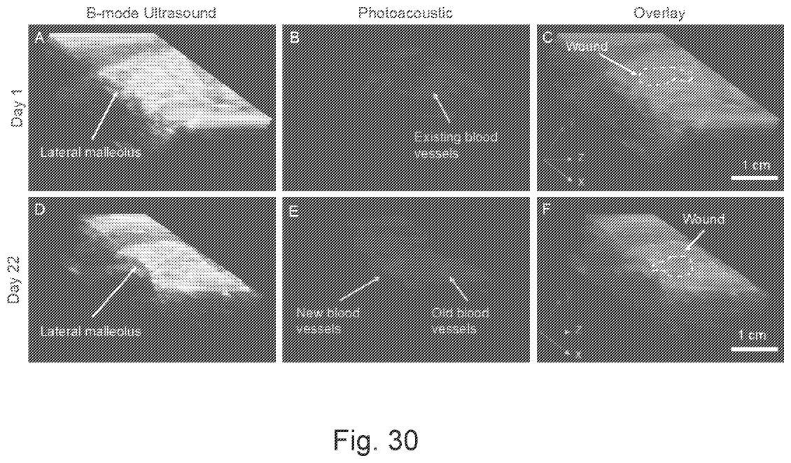

FIGS. 30A-3OF show 3D mappings of angiogenesis. Photoacoustic 3D mappings of the wound bed are shown for patient number (PN) 1. FIGS. 30A-30C show the wounded region on Day 1, illustrating a lack of blood vessels in the wound bed marked by dotted lines in C. A few peripheral blood vessels can be seen on Day 1 in panel B. FIGS. 30D-3OF show the wounded region on Day 22. Newly formed blood vessels can be seen in 30E. Scale bars represents 1 cm.

The particular systems and methods described above have been presented for illustrative purposes and not as a limitation on the subject matter described herein. More generally, in one aspect, a method is presented for monitoring treatment of a wound. In accordance with the method, a photoacoustic ultrasound image of a wound on a patient is obtained. The photoacoustic ultrasound image is processed to extract information that is reflective of a rate of angiogenesis or oxygenation. A degree of wound healing is assessed based at least in part on the extracted information. The wound is treated based at least in part based on the assessed degree of wound healing. The treatment is monitored over time by obtaining and processing additional photoacoustic ultrasound images at subsequent times.

In some embodiments the extracted information that is reflective of a rate of angiogenesis or oxygenation is a measure of intensity of the photoacoustic ultrasound image, the measure of intensity being correlated with a rate of change in tissue angiogenesis or oxygenation.

In some embodiments the treatment of the wound is modified or an additional treatment of the wound is performed based on the monitoring.

In some embodiments the treatment is selected from the group including skin grafts, debridement and hyperbaric therapy.

In some embodiments obtaining a photoacoustic ultrasound image of a wound on a patient includes performing a photoacoustic ultrasound scan of the wound.

In some embodiments the method further includes predicting if the wound is or is not responding to the treatment based on the monitoring.

In some embodiments the method further includes predicting if the wound is or is not responding to the treatment based on a change in the measure of intensity over time.

In some embodiments the predicting is performed within 30 days of initiation of the monitoring.

In some embodiments the predicting further predicts a time needed for the wound to heal.

In some embodiments the method further includes predicting that the wound is responding to the treatment if the measure of intensity indicates that the intensity is increasing over time.

In some embodiments the measure of intensity is a mean gray scale value of the photoacoustic ultrasound image.

In some embodiments the wound is of a type selected from the group including a decubitus ulcer, a diabetic ulcer and an insufficiency injury.

In another aspect of the subject matter described herein, a method for treating a wound is provided. In accordance with the method, a photoacoustic ultrasound image of a wound on a patient is obtained. The photoacoustic ultrasound image is processed to extract information that is correlated to a rate of change in tissue angiogenesis or oxygenation. A degree of wound healing is assessed based at least in part on the extracted information. The wound is treated based at least in part based on the assessed degree of wound healing.

In some embodiments the treatment is selected from the group including skin grafts, debridement and hyperbaric therapy.

In some embodiments the extracted information is a measure of intensity of the photoacoustic ultrasound image.

In some embodiments the measure of intensity is correlated with wound healing.

In some embodiments the measure of intensity is a mean gray scale value of the photoacoustic ultrasound image.

In some embodiments the method further includes monitoring the treatment over time by obtaining and processing additional photoacoustic ultrasound images at subsequent times.

In some embodiments the method further includes determining that the wound is healing if a measure of intensity of the photoacoustic ultrasound image extracted from the additional photoacoustic ultrasound images increases over time.

In some embodiments the method further includes predicting if the wound is or is not responding to the treatment based on the monitoring.

In some embodiments the predicting is performed within 30 days of initiation of the monitoring.

In some embodiments the predicting further predicts a time needed for the wound to heal.

In some embodiments the method further includes predicting if the wound is or is not responding to treatment based on a change in a measure of intensity of the photoacoustic ultrasound image over time.

In some embodiments the method further includes predicting that the wound is responding to treatment if the measure of intensity indicates that the intensity is increasing over time.

In some embodiments obtaining a photoacoustic ultrasound image of a wound on a patient includes performing a photoacoustic ultrasound scan of the wound.

In some embodiments the wound is of a type selected from the group including a decubitus ulcer, a diabetic ulcer and an insufficiency injury.

Aspects of the subject matter described herein, such as the processing of the photoacoustic ultrasound images, ins some case may be described in the general context of computer-executable instructions, such as computer programs, being executed by a processor. Generally, computer programs include routines, programs, objects, components, data structures, and so forth, which perform particular tasks or implement particular abstract data types. Aspects of the subject matter described herein may also be practiced in distributed computing environments where tasks are performed by remote processing devices that are linked through a communications network. In a distributed computing environment, program modules may be located in both local and remote computer storage media including memory storage devices. For instance, some aspects of the claimed subject matter may be implemented as a computer-readable storage medium embedded with a computer executable program, which encompasses a computer program accessible from any computer-readable storage device or storage media. For example, computer readable storage media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick, key drive . . . ). However, computer readable storage media do not include transitory forms of storage such as propagating signals, for example. Of course, those skilled in the art will recognize many modifications may be made to this configuration without departing from the scope or spirit of the claimed subject matter.

1. A method of monitoring treatment of a wound comprising:
obtaining a photoacoustic ultrasound image of a wound on a patient;
processing the photoacoustic ultrasound image to extract information that is reflective of a rate of angiogenesis or oxygenation;
assessing a degree of wound healing based at least in part on the extracted information;
treating the wound based at least in part based on the assessed degree of wound healing; and
monitoring the treatment over time by obtaining and processing additional photoacoustic ultrasound images at subsequent times.

2. The method of claim 1 wherein the extracted information that is reflective of a rate of angiogenesis or oxygenation is a measure of intensity of the photoacoustic ultrasound image, the measure of intensity being correlated with a rate of change in tissue angiogenesis or oxygenation.

3. The method of claim 1 further comprising modifying the treatment of the wound or performing an additional treatment of the wound based on the monitoring.

4. The method of claim 3 wherein the treatment is selected from the group including skin grafts, debridement and hyperbaric therapy.

5. The method of claim 1 wherein obtaining a photoacoustic ultrasound image of a wound on a patient including performing a photoacoustic ultrasound scan of the wound.

6. The method of claim 1 further comprising predicting if the wound is or is not responding to the treatment based on the monitoring.

7. The method of claim 2 further comprising predicting if the wound is or is not responding to the treatment based on a change in the measure of intensity over time.

8. The method of claim 6 wherein the predicting is performed within 30 days of initiation of the monitoring.

9. The method of claim 6 wherein the predicting further predicts a time needed for the wound to heal.

10. The method of claim 7 further comprising predicting that the wound is responding to the treatment if the measure of intensity indicates that the intensity is increasing over time.

11. The method of claim 2 wherein the measure of intensity is a mean gray scale value of the photoacoustic ultrasound image.

12. The method of claim 1 wherein the wound is of a type selected from the group including a decubitus ulcer, a diabetic ulcer and an insufficiency injury.

13. A method for treating a wound, comprising:
obtaining a photoacoustic ultrasound image of a wound on a patient;
processing the photoacoustic ultrasound image to extract information that is correlated to a rate of change in tissue angiogenesis or oxygenation;
assessing a degree of wound healing based at least in part on the extracted information; and
treating the wound based at least in part based on the assessed degree of wound healing.

14. The method of claim 13 wherein the treatment is selected from the group including skin grafts, debridement and hyperbaric therapy.

15. The method of claim 14 wherein the extracted information is a measure of intensity of the photoacoustic ultrasound image.

16. The method of claim 15 wherein the measure of intensity is correlated with wound healing.

17. The method of claim 15 wherein the measure of intensity is a mean gray scale value of the photoacoustic ultrasound image.

18. The method of claim 14 further comprising monitoring the treatment over time by obtaining and processing additional photoacoustic ultrasound images at subsequent times.

19. The method of claim 18 further comprising determining that the wound is healing if a measure of intensity of the photoacoustic ultrasound image extracted from the additional photoacoustic ultrasound images increases over time.

20. The method of claim 18 further comprising predicting if the wound is or is not responding to the treatment based on the monitoring.

21. The method of claim 20 wherein the predicting is performed within 30 days of initiation of the monitoring.

22. The method of claim 21 wherein the predicting further predicts a time needed for the wound to heal.

23. The method of claim 18 further comprising predicting if the wound is or is not responding to treatment based on a change in a measure of intensity of the photoacoustic ultrasound image over time.

24. The method of claim 23 further comprising predicting that the wound is responding to treatment if the measure of intensity indicates that the intensity is increasing over time.

25. The method of claim 13 wherein obtaining a photoacoustic ultrasound image of a wound on a patient including performing an photoacoustic ultrasound scan of the wound.

26. The method of claim 13 wherein the wound is of a type selected from the group including a decubitus ulcer, a diabetic ulcer and an insufficiency injury.
